(19) United States
(12) Patent Application Publication
(10) Pub. No.: US 2014/0236312 A1
(43) Pub. Date: Aug. 21, 2014

(54) CORTICAL BONE SCAFFOLD FOR GUIDED OSTEON REGENERATION IN LOAD-BEARING ORTHOPAEDIC APPLICATIONS

(71) Applicants: Mark R. Appleford, San Antonio, TX (US); Marcello Pilia, San Antonio, TX (US)

(72) Inventors: Mark R. Appleford, San Antonio, TX (US); Marcello Pilia, San Antonio, TX (US)

(21) Appl. No.: 14/116,687

(22) PCT Filed: May 10, 2012
(86) PCT No.: PCT/US12/37312

§ 371 Date: Apr. 25, 2014

Related U.S. Application Data
(60) Provisional application No. 61/484,557, filed on May 10, 2011.

(51) Int. Cl. A61L 27/50; A61F 2/28; A61L 27/56; A61L 27/12

(52) U.S. Cl. CPC A61L 27/50; A61L 27/12; A61F 2/28; A61L 27/56
USPC 623/23.61; 424/602; 264/313; 425/4 R

(57) ABSTRACT

Described is an artificial bone scaffold with an architecture resembling cortical bone, including microchannel-like structures resembling osteons. The scaffold enhances the ability of osteoblasts to secrete organized collagen and mineralize extracellular matrix within the osteon-like channels, thus promoting scaffold strength.

### BACKGROUND OF THE INVENTION

1. Field of the Invention

The invention generally relates to the field of bone tissue engineered scaffolds. More particularly the invention relates to bone scaffolds that can be used to repair load bearing defects by mimicking the structure of natural bone.

2. Description of the Relevant Art

The skeletal system gives humans and vertebrates structural support as well as protection to major organs from injuries. Bones are composed of two different structures: cortical (compact) and trabecular (cancellous). These two different structures can be easily identified by looking at a cross section of long bones. The interior portion of bone is composed of trabecular tissue and resembles the structure of a sponge. It has 75-90% porosity and is often surrounded by a shell of cortical bone. Such high porosity allows for the penetration of blood vessels, nerves, bone marrow, and some mechanical support.

Cortical bone is the major structure responsible for supporting physiological loads. The porosity of this structure, which is between 5 and 10%, is much lower than its trabecular counterpart. Anatomically cortical bone is made up of small units called osteons. These are concentric channel-like structures with diameters that ranges between 40 and 530 μm with an average diameter of 250 μm. To further support the compact density of cortical bone, studies have reported an average of 17.5 osteons/mm² in human femurs. Within each osteon lamellae are found. This structure consists of concentric layers of mineralized bone and extracellular matrix (ECM). Lacunae describe the housing of bone cells trapped between lamellae. The different lamellae communicate to each other through small channels called canaliculi. The void space found within the innermost lamellae allows for nutrient and blood transportation throughout cortical bone and is called a Haversian canal. These structures are connected to each other through small microchannels called Volkmann's canals.

Four types of cells make up bone tissue and are classified accordingly to whether they secrete or break down bone. Tissue-secreting cells are called osteoblasts and bone-resorbing cells are called osteoclasts. As osteoblasts mature, some get trapped in the bone matrix, becoming osteocytes. Others avoid the matrix entanglement because they reside on the bone's surface becoming bone lining cells (BLC). These four cell types work together in remodeling to maintain bone homeostasis.

Osteoblasts are mononuclear cells which are formed when mesenchymal stem cells (MSCs) differentiate. MSCs are found in the stromal tissue of the bone marrow and in the cambium (inner) layer of the periosteal membrane. The main function of osteoblasts is to secrete osteoid. The osteoid is the organic portion of the bone matrix, which is composed of Type-I Collagen (Col-I), noncollagenous proteins, proteoglycans, and water. As osteoids mature they are mineralized by the osteoblasts in locations called calcification points.

Osteoblasts get trapped within newly developed mineralized tissue becoming osteocytes. This type of bone cell resides in the lacunae and communicates with different osteocytes through canaliculi. The anatomy of the osteocytes allows these mature cells to help transfer bone minerals throughout the bone contributing to bone homeostasis. Details of the exact role of osteocytes is still unknown, but researchers believe they are the mechanosensors of bone.

BLCs are similar to osteocytes in that they are both derivatives of osteoblasts. When bone matrix formation ceases, osteoblasts become quiescent and lay flat against the surface of the periosteum becoming BLCs. These cells are also believed to be the mechanosensors as well as chemical sensors of bone. In fact they sense mechanical loads and have receptors for parathyroid hormones, estrogen, and other chemical messengers, which help regulate calcium storage and bone marrow functions.

Osteoclasts are formed by fusion of monocytes creating a multinuclear cell. Their main role is to resorb bone tissue. Osteoclasts do so by breaking down up to tens of microns of bone per day. Specifically, osteoclasts attach to the bone surface and break down bone with the aid of self-secreted acidic enzymes. This environment dissolves the collagen content of bone and with time all minerals, ECM residuals, and excess chemicals are incorporated in secretory vesicles, found within the osteocytes, to be excreted.

ECM is found throughout the bone, particularly in the extracellular spaces of the lacunae and canaliculi. The ECM ties the components of bone together through mineralization. The composition of the ECM is mostly Col-I, non-collagenous proteins, and inorganic salts. Col-I gives bone elasticity while the inorganic salts give it compression strength. The Col-I constituents are secreted by the osteoblasts in the form of amino acids. In the early stage of osteoid formation, these amino acids assemble to form fine collagen fibrils, which become larger with time. In the later stage of osteoid formation, bone minerals are deposited and grow in size until the osteoid is fully developed. It is still not clear whether collagen fibrils are self-assembled or whether they are cell-directed.

During development, while bone is depositing for the first time, the osteonal structure is disordered and the collagen fibrils are fully oriented. This early structure is also known as woven bone. Later in development bone responds to mechanical load. This partially leads to continual tissue remodeling. This is one of the process in which lamellar bone is formed. Lamellar bone is characterized by collagen assuming parallel orientation and the embedding of secondary osteons. Moreover, in lamellar bone the preexisting matrix is resorbed and a cement line is laid down signaling osteoblasts location for attachment.

Three types of secondary osteons are found in lamellar bone: dark, bright, and intermediate. Osteonal ECM orientation in lamellar bone can be evaluated under polarized light. Dark osteons appear dark under polarized light due to their inability to rotate the plane of light in respect to the plane of the section. These osteons describe collagen fibers that run parallel to the long axis of the osteon. Bright osteons rotate the plane of polarized light appearing bright under polarized microscope. These osteons are characterized by collagen fibers running in a transverse spiral direction to the long axis of bone. The third osteon type is intermediate or alternating and is a combination of bright and dark osteons in succession. Not all researchers believe that the collagen fibril orientation just described is responsible for this alternating structure. In fact some scientists believe that the change in the brightness of osteons under polarized light is due to different densities of the ECM elementary components. That means that the lamellae alternate between collagen and cementing mineral substances.

Segmental bone defects (SBD) are a common problem in medicine and orthopedics because they do not heal on their own. The gold standard approach favored by surgeons to overcome this problem is through the use of an autograft which is the implantation of a homologous section of bone tissue. However, many problems are associated with this technique, including infections and immune rejection.

The advent of bone tissue engineering has brought new ideas, discovery/development of new biomaterials, and the characterization of these materials for bone tissue engineering purposes. Tissue engineering scaffolds for SBD should promote vascularity while maintaining the structural and mechanical integrity of the natural tissue. Bone tissue engineering approaches include cell-based, scaffold-based, and delivery-based strategies. Regardless of the approach that is used, different scaffolding materials are now used in conjunction with stem cells, drugs, and growth factor delivery to promote bone regeneration.

Although several approaches are able to promote vascularity, mechanical stability is still a weak component of these implants even though it is crucial to the success of the implant. Most current scaffolds are not strong enough to withstand the person's own weight and are easily crushed under small loads. This problem has led to temporary metal plate implants, used to divert the load away from the scaffolds, becoming a necessity to be placed in conjunction with the SBD scaffolds. Unfortunately these plates lead to stress shielding effects around the defect area, slowing the healing response and promoting bone resorption at the implant.

Not many studies have been conducted to analyze the effect of substrate curvature on cell attachment, orientation and growth. There are few studies that have focused on the strength of cytoskeletal filaments' attachment on curved substrates to determine cell membrane deformation and cell motility direction. Trabecular and cortical bone offer anatomically different substrate curvatures to bone cells for attachment. The shape of the trabeculae, the long filament-like structures that make up trabecular bone, resemble a cylinder-like structure. When bone cells attach to its surface they are exposed to a convex curvature. On the other hand, the osteons' shapes resemble that of long microchannels and when bone cells attach to the osteon they assume the shape of a concave curvature. Limited literature is available to show the difference curvature plays in ECM secretion or stress, factor activation.

The inorganic minerals that make up bone ECM fall into the category of calcium phosphates (CaP). There are many CaP substrates that simulate the properties of bone. The nature of these materials has increased their use as grafts for bone repair, augmentation or substitution. CaPs differ from one another in origin, composition, morphology, and physicochemical properties. CaPs also share outstanding properties that elevate these ceramics to ideal biomaterials, such as biocompatibility, osteoconductivity and bioactivity. CaPs are biocompatible because they do not trigger the body's immune response and do not cause rejection after implantation. The osteoconductivity property of CaPs supports tissue ingrowth, osteoprogenitor cell growth, and the development of bone formation by promoting the attachment, proliferation, differentiation, and migration of bone cells. CaPs can also be considered bioactive as they develop a direct, adherent, and strong bond with the bone tissue and mediate an exchange of calcium and phosphorous ions between cell matrix and substrate. In bone tissue engineering, the structural, morphological or chemical aspect of CaP can be modified to best suit the research interest. The biggest factors that play a role in osteoconduction are porosity, degradation, 3D environment and surface properties. In particular, researchers have proven that pores of different sizes promote cell attachment and bone formation as well as the creation of vasculature and possibly osteons. The more porous the scaffold, the more surface area the organism is in contact with. However, the more porous the scaffold also corresponds with weaker compression strength of the implant.

There are many different types of CaP: Hydroxyapatite (HAp), β-Tricalcium Phosphate (β-TCP), bioglass, alumina and various combinations of these. Naturally available CaPs are coralline HAp and bovine-derived apatites. However, synthetic CaPs are the most common and include HAp, β-TCP, and biphasic CaPs. HAp and β-TCP are the most commonly used/commercialized ceramics for biomedical applications.

HAp is one of the strongest CaP materials and although its compressive strength is much higher than bone (350 MPa vs. 180 MPa respectively), the porosity and architecture that favor osteoconductivity make it only as strong as trabecular bone. However, when HAp is combined with tissue ingrowth, the compression strength raises proportionally. This material can be manipulated in many different shapes and its micro-sized particles are well accepted by the body. This material's downside includes brittleness and its resorption rate. As a ceramic, brittleness is a common disadvantage of these types of materials. The resorption rate is so low that an HAp implant can remain in the body for more than 10 years. Thus, HAp geometry and architecture needs to be properly characterized to promote and support bone regeneration, bone marrow formation, blood supply and osteogenesis.

Tricalcium Phosphate (TCP) can be produced having two different crystal structures: α and β. The α configuration has a polygon shape while the β configuration is spherical and can be packed more closely. This property favors β-TCP in most biomedical applications. β-TCP's compressive strength is not as high as HAp, and its compression strength when modeled with osteoconductive architecture is similar to that of trabecular bone (12 MPa). Like all other ceramics it is brittle and also weak under tensile and shear stress. The true difference between β-TCP and HAp is resorption time which, for β-TCP, is counted in months instead of years (6-18 months). β-TCP is resorbed by osteoclasts and in doing so it does not cause any inflammatory and/or giant cell responses. This can change if the ratio of β-TCP resorption is too high.

The development of microchannels in CaP materials has been previously reported with the aim to create an interconnected pore structure, but it was never investigated on the basis for osteonal regeneration. In two studies microchannels where recreated from the burnout of polymeric fibers during sintering. In one study, an osteon-like bone growth in the microchannels was seen after 4 weeks implantation in rabbit tibia. In another study, lamellar-like structures were seen growing in channel pores of roughly 300 μm diameter as well as an increase in compression strength.

Researchers are working on a solution to develop a load-bearing scaffold that will allow individuals to recover from their injuries in a timely manner. Many current scaffolds have an architecture which resembles trabecular bone; scaffolds mimicking the structure of cortical bone are virtually unseen in the field. It would be desirable to develop a load bearing scaffold model that can mimic the natural structure of bone, giving stem cells the necessary environment to promote growth, strength and organization.

### SUMMARY OF THE INVENTION

In one embodiment, a bone scaffold for the repair of load-bearing bone damage includes hydroxyapatite and β-tricalcium phosphate. The scaffold may have an architecture that matches the structure of cortical bone. For example, the scaffold may have longitudinal microchannels that recreate the structure of secondary osteons. The scaffold may also have a high interconnectivity between these microchannels to recreate the structure of Volkman's canals, allowing blood and nutrients to be moved within the scaffold. In some embodiments, the amount of β-tricalcium phosphate present in the scaffold is preselected to cause the scaffold to be resorbed in at least a year. To promote bone repair, stem cells may be coupled to the scaffold.

A method of forming a scaffold for the repair of load-bearing bone damage includes forming a mold having a shape that is complementary to the bone repair site; coupling wires to the mold; placing a fluid composition of hydroxyapatite and β-tricalcium phosphate in the mold; and removing the wires from the mold. Specifically, a mold having an interior space that is complementary to the bone repair site may be obtained. Wires (metal or polymeric) are coupled to the mold such that the wires extend through an interior space of the mold. A fluid composition of hydroxyapatite and β-tricalcium phosphate is placed into the interior space of the mold; wherein the fluid composition surrounds at least a portion of the wires. After the fluid composition hardens the wires are removed from the mold.

In some embodiments, the wires are formed from a polymeric material such that the polymeric wires are removed by heating the scaffold to a temperature sufficient to decompose the wires. Metal wires will be pulled out just before the sintering process of the ceramic scaffold. To inhibit microchannel curvature, the wires may be secured to the mold, such that the position of the wires does not substantially change when the fluid composition is added to the interior space. To further define the interior of the scaffold, a porous material (e.g., a polymeric sponge (e.g., polyurethane)) may be placed in the interior space prior to coupling the wires to the mold, wherein the wires pass through the porous material when the wires are coupled to the mold. In some embodiments, the fluid composition further comprises particles of an organic material (e.g., sucrose particles), wherein the method further comprises heating the scaffold to a temperature sufficient to decompose the particles of organic material. In some embodiments, the particles are nanoparticles that, after decomposition, create nanopores in the formed scaffold.

In some embodiments, the mold may be subjected to sonication, after the fluid composition has been placed in the interior space of the mold. The sonication treatment may help to improve the dispersion of the fluid composition within the mold.

A mold for forming a bone scaffold for the repair of load-bearing bone damage, may include a body having an interior surface, wherein the interior surface defines the shape of the scaffold; a top plate and a bottom plate, couplable to a top end and a bottom end of the body, wherein the top plate and the bottom plate each comprise a plurality of openings, wherein, during formation of the scaffold, one of more wires are placed in the openings. The mold may also include a middle rod, couplable to the top plate and/or the bottom plate, wherein the middle rod defines a hollow portion of the scaffold being formed by use of the mold. One or more alignment rods, extending from the bottom plate, through the body, to a top plate of the mold may be present to allow alignment of the components of the mold. In some embodiments, the top plate and/or the bottom plate are movably positioned with respect to the body, by sliding the plates along the alignment rods.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a mold used to form a scaffold;

FIG. 2A depicts a projection cross-section view of an embodiment of a mold used to form a bone scaffold;

FIG. 2B depicts an embodiment of a main body of the mold of FIG. 2A;

FIG. 2C depicts an embodiment of a bottom plate of the mold of FIG. 2A;

FIG. 2D depicts an embodiment of atop plate of the mold of FIG. 2A

FIG. 3 depicts processing steps for making a bone scaffold;

FIG. 4 depicts a schematic diagram showing morphological measurements for a sectioned scaffold;

FIG. 5 depict SEM photographs of various sectioned bone scaffolds;

FIG. 6 depicts the proliferation rate of HEPM & HFOB in various mediums;

FIG. 7 depicts the ALP activity of HEPM & HFOB in various mediums;

FIG. 8 depicts a schematic diagram depicting a method of determining the angle of orientation of the cells with respect to the microchannel direction;

FIG. 9A depicts a histogram of the DNA assay results for various scaffolds;

FIGS. 9B-9E depict histograms with the differentiation markers findings;

FIG. 9F depicts quantification of Col-I fluorescence in various scaffolds;

FIG. 10 depicts the frequency distribution of the cell orientation in the different scaffolds;

FIG. 11 depicts a schematic diagram of a bioreactor for testing fluid flow rates;

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

In one embodiment, concave substrates of different sizes may be formed on a Hydroxyapatite (HAp) disk that resemble the longitudinal section of osteonal microchannels to study ECM secretion and mineralization. HAp was chosen for this platform due to its biocompatibility both in vitro and in vivo as well as its ability to be casted into different shapes. The first step is to create molds into which the HAp will be casted. The templates to be created are a 2-dimensional (2D) representation of the change in lamellae curvature inside an osteon. Different size molds may be used according to the natural range of osteon size in human long bones, ranging from 50 to 500 μm. Accordingly, in some embodiments, three different size substrates may be built: 50, 250, and 500 μm wavelength. These molds are manufactured to be precise and made of a long-lasting material that will not change shape or deform over time. Additionally, these templates are reproducible so that multiple HAp disks will share the same surface geometry, wavelength and amplitude. In an embodiment, by creating precise, non-deformable molds, HAp may be shaped into specific substrates that resemble different lamellae curvatures.

In one embodiment, HAp disks may be manufactured using drilling and/or pressing techniques. Templates may be made of dental plaster, dental cement, PTFE®, copper, or stainless steel. The combination of casting on a stainless steel template provided accurate and reproducible structures.

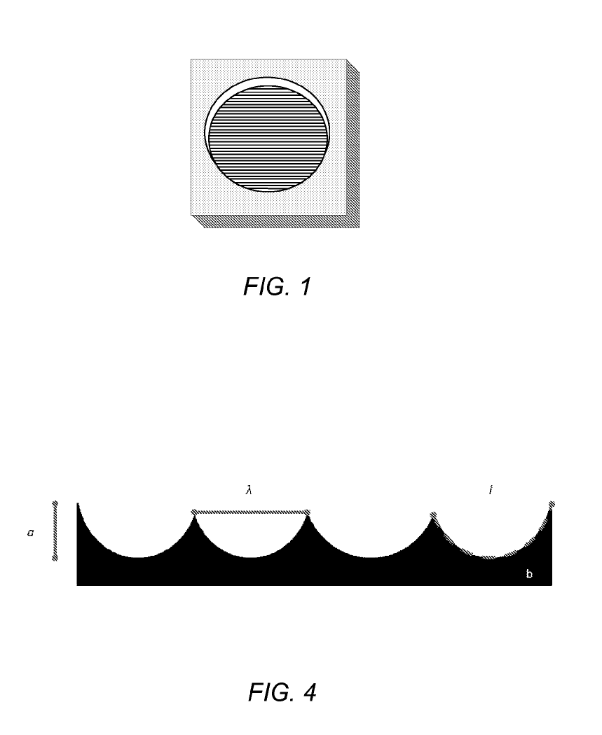

In one embodiment, a template may be created on a methylmethacrylate microscope slide. On each side of the slide a known size wire (e.g., between about 50 μm and 500 μm) is wrapped around with no overlap and no spacing in between. Templates may be made using any type of metal. Examples of metals that may be used include, but are not limited to copper wire (e.g., copper wire available from McMaster-Carr—Atlanta, Ga.) or stainless steel wire (e.g., stainless steel wire available from HM Wire International Inc.—Canton, Ohio). Dental stone cement (e.g., Coecal® Type III Dental Stone—GC America Inc, Alsip, Ill.) is mixed with water to create molds into which HAp slurry could be casted. Each cement mold was about 5 mm tall. Generally, molds may have a depth of from 1 mm to 50 mm. Subsequently a hole (φ=10 mm, but could be of any size) was drilled in the middle of the molds. The cement mold was then placed directly above the wire and was held securely in place by rubber bands. FIG. 1 shows a projection view of a basic mold for making a template. The shape of the curvature and the size of the templates is based on lamellar curvature and its physiological size range in osteons.

The HAp disks may be made by solution casting. An HAp slurry was created by using the process described in Appleford, M. R., et al., “Effects of trabecular calcium phosphate scaffolds on stress signaling in osteoblast precursor cells.” Biomaterials, 2007, 28(17): p. 2747-2753, which is incorporated herein by reference. Briefly, the binders used to stabilize the slurry structure included 3% high molecular weight polyvinyl alcohol, 1% v/v carboxymethylcellulose, 1% v/v ammonium polyacrylate dispersant, and 3% v/v N,N-dimethylformamide drying agent. This solution may then be poured into the molds, dried and sintered. In one embodiment, a sintering process will heat the disks to 1,230° C. by increasing the temperature at 5° C./min and then will rest at this temperature for 5 hours. The sintered disks will then be cooled at a rate of 5° C./min until room temperature is achieved in the furnace (Thermolyne, Dubuque, Iowa).

A ceramic scaffold may be used to recreate the natural osteonal architecture of bone and its original strength. Specifically a hydroxyapatite/tricalcium phosphate (HAp/TCP) composite scaffold may be used to heal critical size load-bearing defects resembling the structure of cortical bone and naturally occurring osteons

In one embodiment, a scaffold with the same physical structure of the naturally occurring osteon may be formed using a blend of hydroxyapatite and tricalcium phosphate (HAp/TCP). Microchannels in the scaffolds may be formed having a diameter of ranging from about 50 μm to about 500 μm. A typical channel will have a 250 μm diameter, the physiological average size osteon. A 60:40 blend of HAp/TCP may be used, however, different blend concentrations may also be considered to match physiological bone resorption anywhere from 100:0 to 0:100 HAp:TCP. Representative ratios that may be used include, but are not limited to, 80:20, 50:50, 40:60, and 20:80 HAp:TCP. Furthermore, porosity may be created in the scaffold by adding nano size sucrose particles that will burn off during sintering. In some embodiments, a porosity of about 67% is used, however other porosities, ranging from 30% to 90% may also be used. For the scaffold to be successful at simulating physiological and mechanical properties of real bone the scaffold may be casted to recreate a dense arrangement of uniform microchannels similar to naturally occurring osteons. The scaffold may be designed to maximize its mechanical strength while promoting microchannel interconnectivity. The TCP present in the scaffold may be completely resorbed within a year, which will match both bone formation and ECM secretion/mineralization by osteoblasts, improving the scaffold's load bearing strength. Meanwhile the pore interconnectivity will promote nutrient distribution within the platform.

When bone marrow derived stem cells are seeded into the newly created HAp/TCP scaffold, they may manufacture organized ECM and strengthen the overall construct. Specifically, the structural osteonal organization of the cells into the microchannels may directly translate into an increase of compression strength of the scaffold. The cells accept the osteonal platform and will attach, proliferate, and differentiate as well as secrete and mineralize their own ECM environment recreating artificial cortical bone. In one embodiment, a HAp/TCP scaffold, when seeded with bone cells or implanted, provides a guide that promotes secretion and mineralization of organized ECM, recreating the natural environment of cortical bone. As the cells continue to secrete and process the ECM in this newly developed scaffold, the implant will gain mechanical strength and the infiltration rate will increase.

In an embodiment, HAp/TCP will be made into a wet slurry that can be casted into molds. To make a batch size of 5 grams HAp/TCP, start by adding 20 ml DI water in a 100 mL beaker, add a stir bar and stir at 300-400 rpm. Add 5% by weight of high molecular weight polyvinyl alcohol (PVA). Increase the temperature to 150° C., wait roughly 15 minutes, and let cool down by separating from hot surface using a clamp. Add 5% weight carboxymethyl cellulose (CMC), wait 10 minutes, and allow cooling to room temperature. Add 3% weight polyethylenimine, and 10% weight dimethylformamide. When the solution is completely clear, add the predetermine ratio of HAp and TCP. Sonicate for 30 minutes to create a uniform distribution. Return to the hot plate, turn the heat to 100° C. to reduce liquid from 20 ml to ˜10 ml.

For the scaffold to be successful at simulating physiological and mechanical properties of real bone the scaffold is casted to recreate a dense arrangement of uniform microchannels similar to naturally occurring osteons. The microchannels in the scaffolds are created to have a diameter of 250 μm, but may also have a diameter from about 50 μm to about 500 μm. The scaffold with the microchannels may be created using specific molds into which the previously described HAp/TCP slurry will be casted.

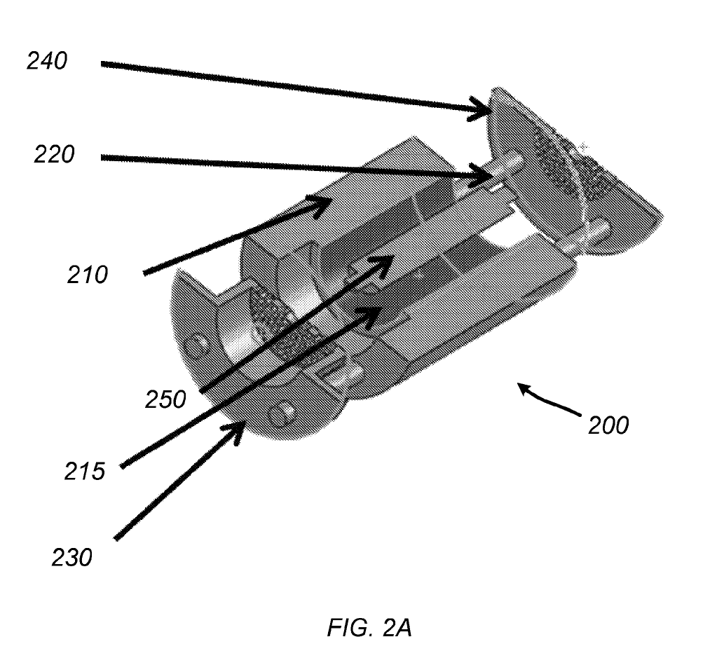

An embodiment of a mold 200 is depicted in FIG. 2A. Mold 200 gives the scaffold a doughnut shape, with polyurethane wire or metal wire or metal rods that may recreate microchannels running longitudinally through the entire scaffold. The perforated ends may be made of metal. Mold 200 may be formed from eight different pieces. A main block 210 is a generally cylindrical block that includes a plurality of alignment holes 212 (depicted in FIG. 2B) that may receive alignment rods 220. The main block 210 may be sectioned into two identical pieces to make the casting of the calcium phosphate easier. Main block 210 has a central hollow interior space 215 in which the scaffold is molded. The main block can be made of dental cement, or any ceramic, metal or polymeric material. The measurements are not necessarily always the same and can change based on the application. The diameter of the interior space 215 in main block 210 can range between 5 mm and 50 mm and the block can be as short as 5 mm and as long as 100 mm. A projection view of the main block is depicted in FIG. 2B.

Mold 200 also includes a top plate 230 and bottom plate 240. Projection views of bottom plate 240 and top plate 230, are depicted in FIGS. 2C and 2D respectively. Top plate 230 and bottom plate 240 include a plurality of openings 245 through which wires (polymeric or metal) may be passed through to create microchannels in the formed scaffold. Top plate 230 and bottom plate 240 may have a diameter of about 5 mm to about 50 mm. Top plate 230 and bottom plate 240 may be formed from a ceramic, metal, or polymeric material. Openings 245 may be machined in the plates using small hole electrical discharge machining, or other methods. Top plate 230 and bottom plate 240 may include alignment openings 248 that align the plates with the main body using alignment rods 220.

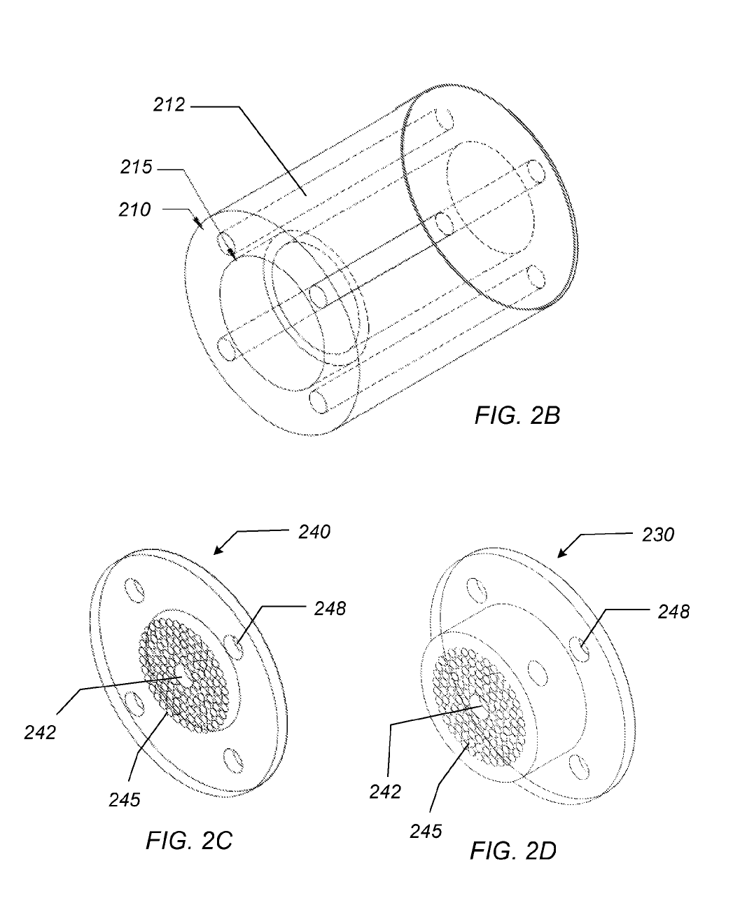

In some embodiments, a scaffold is formed in a hollow cylindrical shape. The hollow portion of the scaffold is formed by placing a middle rod 250, in the interior space of the mold. Middle rod 250 may be coupled to openings 242 formed in bottom plate 240 and/or top plate 230. The middle rod's diameter is typically a percentage of the diameter of the interior space 215 of the mold. The middle rod is optional, but when present, can occupy up to about 95% of interior space 215, varying as a percentage of the volume from 0 to 95%. Details of the scaffold-making process are depicted in FIG. 3. In step (1) the bottom piece of the mold is locked to the main mold, fixing the middle pin in place (2). A polyurethane sponge (20-100 pores per inch) is punched with the same shape of the final scaffold. The precut sponge is placed inside the main chamber (3), Then the top piece is slide into the main assembly and wire is passed through the top and bottom hole chamber (4). At this point the HAp/TCP slurry is placed inside the chamber from the top or the bottom (5). When the chamber is full, proceed to slide the top piece until tight on the main assembly (6). Once the wires have been fed through the mold, they may be clamped at both ends and placed under tension to prevent microchannel curvature.

The entire mold is then moved to a sonicator for two hours to evenly distribute liquid ceramic around the microchannels, placed in the freezer for twelve hours, and placed into a lyophilizer for four days to remove all water. At the end of the four days, the polyurethane wire is cut as close as possible to the molds, and the two ends of the molds are separated. If the molds are difficult to separate, they may be placed in the lyophilizer for one additional day or until all remaining moisture is removed. Once the scaffold is pulled from the molds, the scaffold is placed in a furnace and sintered. The sintering process specifically will involve heating the scaffolds to 1,230° C. by increasing the temperature at 5° C./min and then resting at this temperature for 5 hours. The sintered scaffold is cooled at a rate of 5° C./min until room temperature is reached.

In some embodiments disks may be coated with carbon and the HAp crystal size may be determined using a scanning electron microscope (SEM). In some embodiments, the roughness average (Ra) profile may be determined using a profilometer. Equation 1 below shows how to mathematically determine the roughness average of a surface. Briefly, it is found by measuring the distance between peaks and valleys on the surface and the standard deviation from the center line.

$${R}_{a } = \frac{1}{MN} {\sum }_{K = 0}^{M - 1} {\sum }_{l = 0}^{N - 1} \vert z ( {x}_{k} , {y}_{l} ) \vert \qquad ( Equation 1 )$$

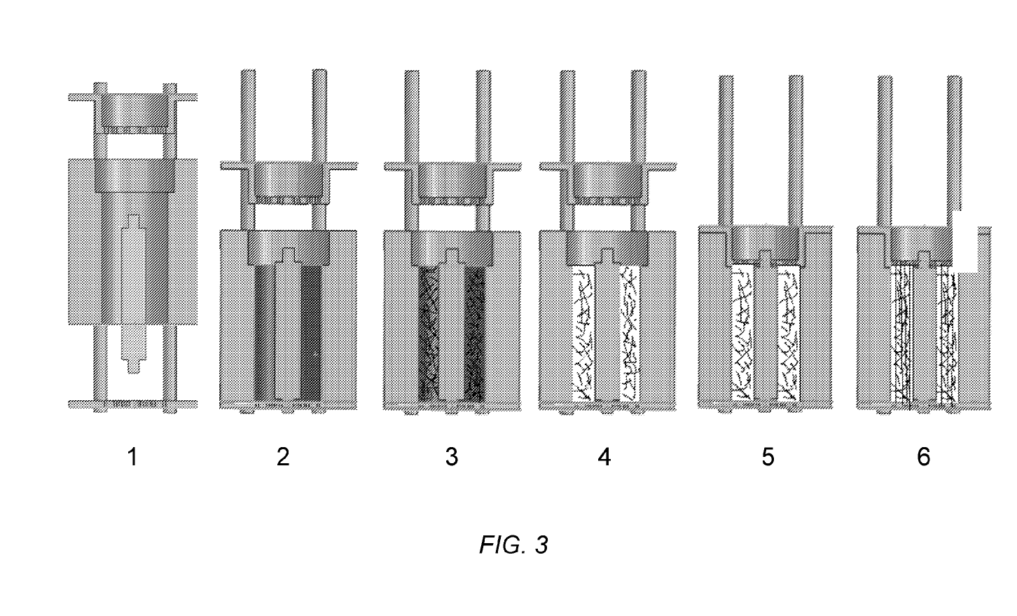

Disks may be analyzed for template geometry and architecture using imaging techniques. In one embodiment, disks will be placed into Peel-a-way® Disposable Embedding Molds (S-22, Warrington, Pa.). These molds may be filled with Pelco® Fast Curing Epoxy Hardener (TedPella Inc, Redding, Calif.) and allowed to harden in air for 24 hours. When the epoxy has hardened completely, the plastic molds may be peeled away and solid epoxy blocks with the template HA disks embedded in it will remain. A microtome saw (Leica SP1600; Wetzlar, Germany) may be used to cut the sample in a direction perpendicular to the template channels. Each cut may be roughly 150 μm thick and will be polished on both sides using a wet-sander (Struers LaboPol-5, 800→1200 grit sand paper; Cleveland, Ohio). Each section may be taken to a microscope (Leica DMIL LED, Wetzlar, Germany), where the dimensions of X (wavelength), a (amplitude), l (arc length), and d_{d} (diameter of the disk) may be acquired using a computer software (Bioquant Osteo 2010, Nashville, Tenn.). FIG. 4 depicts the measurements that are taken from each sectioned sample for testing.

The measurements acquired may then be used to calculate the surface area of each disk. The number of microchannels (n) present in disk template may be determined from Equation 2. The diameter of the stretched out channels (dₑ) may then be calculated with Equation 3 and finally, Equation 4 may be used to determine the surface area of disks (Aₑ) when they are stretched in the geometrical shape of an ellipse.

$$\begin{matrix}n = \frac{{d}_{d}}{λ} & ( Equation 2 ) \\ {d}_{e} = n \times l & ( Equation 3 ) \\ {A}_{e} = π \frac{{d}_{d}}{2} \frac{{d}_{e}}{2 } & ( Equation 4 )\end{matrix}$$

Table 1 shows results of morphology characterization of the HAp disks. All measurements were taken after thin histological longitudinal sections of the disks were cut, polished, and analyzed using Bioquant Osteo®. All values reported with standard error of the mean (SEM).

TABLE 1

| Template | | | | Area |
|---|---|---|---|---|
| (μm) | l (μm) | a (μm) | l (μm) | (mm²) |
| 75 | 60.44 + 6.49 | 26.22 + 9.11 | 90.35 + 21.38 | 51.06 + |
| | | | | 11.16 |
| 500 | 368.59 + 30.59 | 152.27 + 24.46 | 545.14 + 65.96 | 50.64 + |
| | | | | 5.43 |

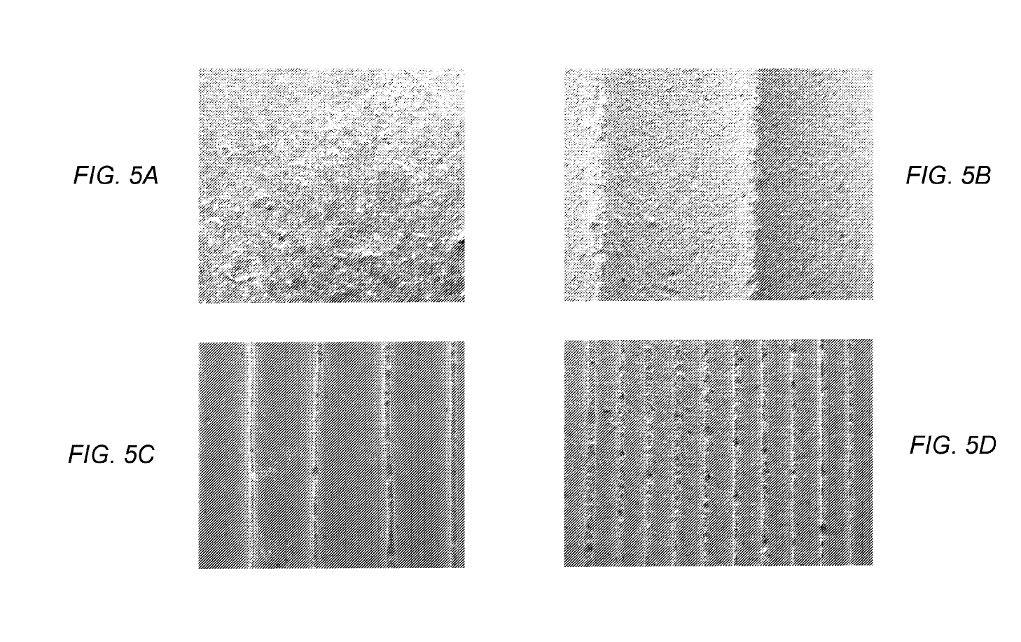

FIG. 5 shows 100× Scanning Electron Microscope pictures of 4 different templates tested. FIG. 5A depicts a scan of a flat disk, FIG. 58 depicts a scan from the 500 μm group, FIG. 5C depicts a scan from the 250 μm group, and d) FIG. 5D depicts a scan from the 50 μm.

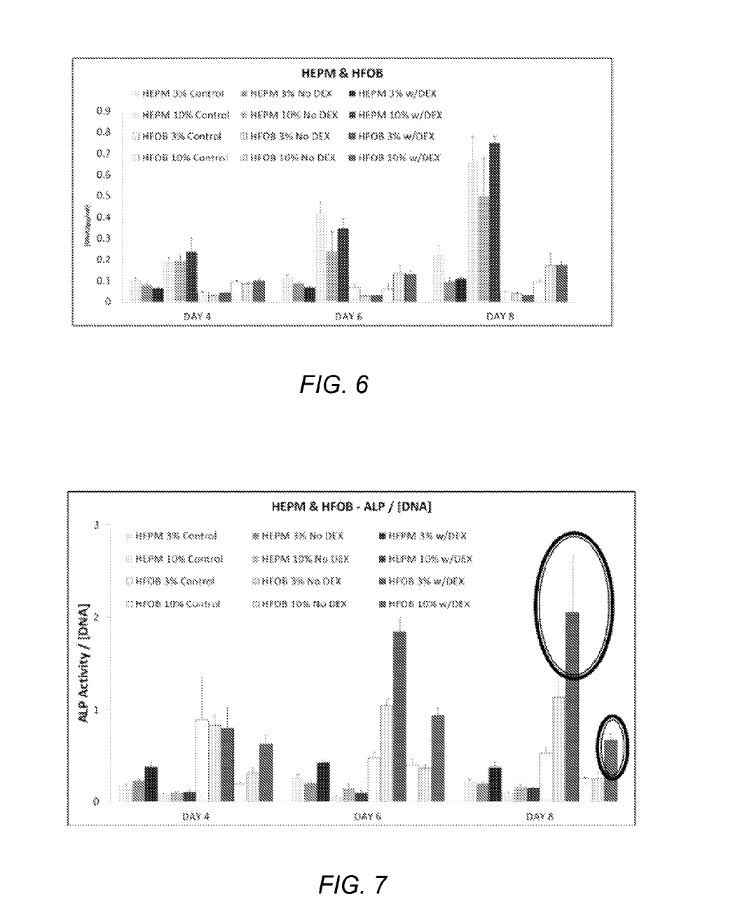

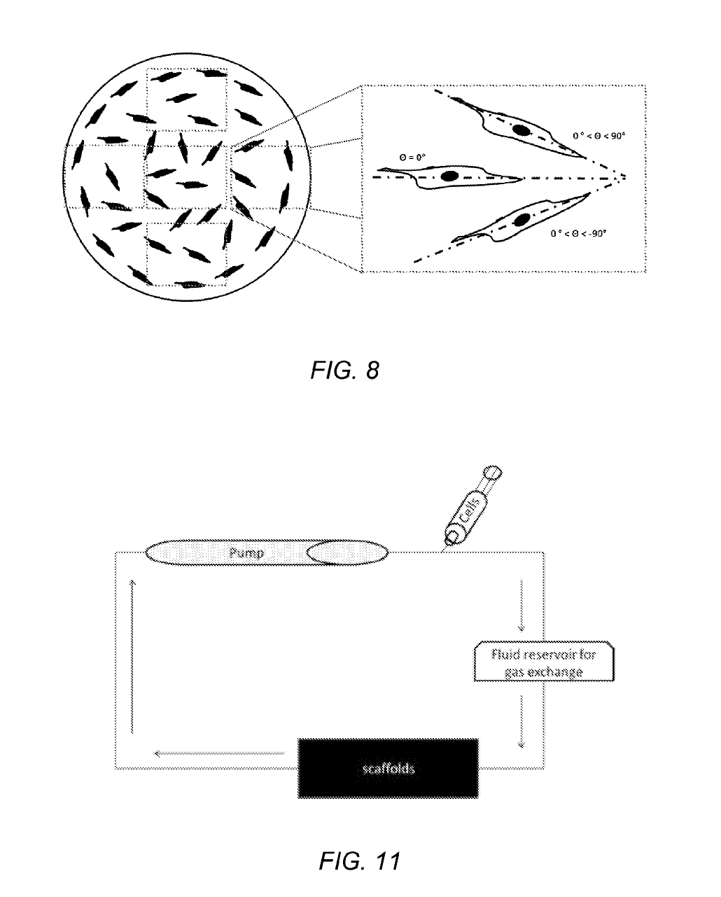

ECM and specifically type-I collagen (Col-I) secretion by osteoblasts may be achieved using the created disks. Cells that are precursors of early bone lineage were tested on the created disks. In some embodiments, it is also useful to give the cells the necessary stimuli to favor differentiation over proliferation. Cell lines that may be used in these experiments include Human Embryonic Palatal Mesenchymal stem cells (HEPMs), Human Fetal Osteoblasts, or bone marrow-derived mesenchymal stem cells (MSCs). All of these cell lines are bone progenitor stem cells, yet HEPMs are more pluripotent than HFOBs. In fact, HEPMs can differentiate into cartilage, bone, fat, and bone marrow cells. HFOBs instead can only differentiate into bone. The two cell lines were compared in their ability to differentiate and activate Alkaline Phosphatase (ALP), a soluble enzyme expressed and secreted by active osteoblasts. While selecting between cell lines, different growth/differentiation media compositions were tested. Precisely, each cell line was exposed to 6 different types of media: Dulbecco's Modified Eagle Medium (DMEM) with either 3% or 10% fetal bovine serum (FBS) alone (control), 3% or 10% FBS with 10 mM O-Glycerine Phosphate and 50 μg/mL Ascorbic acid, and 3 or 10% FBS with 10 mM β-Glycerine Phosphate, 50 μg/mL Ascorbic acid, and 10-8 M Dexamethasone. The study lasted 8 days and it consisted of four time points: 2, 4, 6, and 8 days. The cell lysates were then analyzed for proliferation using a Quant-iT™ PicoGreen® assay by Invitrogen, and the ALP was detected using an Alkaline Phosphatase Fluorescent Detection Kit (Sigma). ALP results were normalized to DNA. The results proved that the HFOBs grown in a 3% FBS and Dexamethasone media differentiated the most by activating the most ALP. The results of this experiment are shown in FIGS. 6 and 7. By measuring the DNA results (FIG. 6), we observed that the 10% FBS media shows a trend of increased proliferation when compared to the 3% groups. Also, it is clear that the HEPM cell line proliferated much faster than the HFOB line with either media composition. There was no statistical difference seen in the amount of proliferation between osteogenic and proliferating media of same percentage FBS. In the second graph (FIG. 7), which shows the ALP activity levels standardized to DNA content of each cell, we can see that although HEPMs proliferated considerably, they did not activate high levels of ALP. However, the HFOB cell line, when exposed to 3% FBS and osteogenic media with DEX (groups circled in black) was able to activate the highest levels of ALP.

The same HFOBs from above were used for preliminary attachment/morphology studies. Primary HFOBs were brought up according to vendor instructions in T-75 flasks. Once confluence was reached, the cells were exposed to Trypsin for 10 minutes at 37 C. Cells in solution were counted using a cell counter (Z2 Coulter® Particle Count and Size Analyzer; Beckman Coulter™—Brea, Calif.), and the cytoskeleton of the cells was stained using Vybrant® Dil cell-labeling solution (Molecular Probes; Eugene, Oreg.). The controls in this study were a flat HAp disk and cells growing alone in a well. The two groups tested were the 75 and the 500 μm HAp disk templates. At the time of the experiment the disks had not been characterized and the cells were seeded at over confluence. The disks were used to take fluorescent pictures at 2, 4, 6, and 8 days. In the tests the cells organized in clusters along the 75 μm microchannels. The cells were over seeded on the template and cell morphology could not be determined. Cells also organized in the 500 μm microchannels. The cells prefer to attach to the bottom of the template between peaks.

2D Disks may be used to test cell proliferation, differentiation, ECM production/mineralization, cell orientation, and mechanical ECM characterization of local strengths, toughness and stiffness.

### Human Fetal Osteoblast Cell Culture

To test the interaction between the newly developed disks and osteoblast precursor cells, Human Fetal Osteoblasts (HFOb) cells (Cat. #406-05f, Cell Applications, Inc.) were used. The choice of cell was due to previous unpublished data that compared HFOB and human epithelial palatal mesenchyme (HEPM) stem cells in the time needed to activate alkaline phosphatase (ALP) and the activity level each cell line expressed under osteogenic media. HFOb showed earlier and higher amounts of ALP, activity throughout the 12 day study. The cells were cultured in growth media containing Dubecco Modified Eagle Medium (DMEM), 10% Fetal Bovine Serum (FBS), and 1% Penicillin Streptomycin Amphotericin B Solution (PSA) (all purchased from Invitrogen, USA). When cells reached confluence on the cell culture-flask, the HFOb were washed with phosphate buffered saline (PBS) and then 0.25% Trypsin/EDTA in osteogenic media (DMEM, 3% FBS, 1% PSA, 10 mM Glycerolphosphate, 50 μg/mL Ascorbic acid and 10 nM Dexamethasone). The cells in solution were counted (Z₂ Coulter® Particle Count and Size Analyzer; Beckman Coulter™—Brea, Calif.) and seeded on the disks at a density of 100 k/disk. The experiment had four time points tested: 6, 12, 18 and 24 days (n=12). For n=8 disks, media was collected and then each disk was washed with PBS, followed by cell permeabilization using 0.1% Triton X-100 in PBS (PBS-T), which was added and supernatant collected after processing through one freeze/thaw cycle. The remaining n=4 disks were immersed in 4% formaldehyde for imaging.

Cell proliferation was measured directly from the cell lysate solution. Specifically, 25 μL of lysate was added to the Quant-iT™ PicoGreen® dsDNA kit (Invitrogen, USA). This assay was performed in black opaque 96 well plates and the fluorescence was assessed using a Synergy 2 microplate reader (Biotek Synergy 2—Winooski, Vt.). The plate was excited at 485/20 nm, and the emitted light was measured at 528/20 nm.

HFOb differentiation was determined by testing the cell lysates for bone-specific transcription factor runt-related transcription factor 2 (RUNX2), Alkaline Phosphatase (ALP), Dental Matrix Protein 1 (DMP1), and Osteopontin (OPN) activity. RUNX2 activity was measured from the lysate using an indirect enzyme linked immunoabsorbent assay (ELISA). Specifically, 50 μL of lysate were pipetted into a protein-attachment ready microplate and diluted in PBS-T solution at a ratio of 1:1. After 24 hours the wells were quenched in 0.6% H₂O₂, and blocked in 10% fetal bovine serum (FBS). Anti-RUNX2 primary antibody (Cat #41-1400, Invitrogen) was then added overnight using a concentration of 1 μg/mL, followed by PBS-T washes and a secondary antibody (Cat #81-6720, Invitrogen) for one hour using a 1/7′ 500 dilution. Pierce 1-step ultra TMB was added to each well and the reaction was stopped using 2M H₂SO₄. The plate absorbance was measured using the same microplate reader used in the DNA analysis. The absorbance was read at 450 nm with reference at 655 nm. Primary to secondary ratio was optimized after performing an unpublished signal-to-noise ratio. ALP activity was also assessed from the cell lysate using an ALP Fluorescence Detection Kit (APF, Sigma-Aldrich). Precisely, 10 μL of lysate were added in black opaque 96 well plates. The fluorescence was read after exactly 45 minutes with an excitation of 360 nm, and an emission of 460 nm. DMP1 was detected and quantified using the same PACE technique used for the RUNX2 assay. All of the steps remained the same with the difference that the primary antibody used was anti-DMP1 (code ab76632, Abcam) at a concentration of 2.5 μg/mL, and the secondary antibody used was the same used in the RUNX2 and was diluted 1/5′ 000. Absorbance readings remained the same as well. As in RUNX2, primary to secondary, antibody ratio was optimized after performing an unpublished signal-to-noise ratio. OPN detection from the cell lysate solution was performed using the bone panel Milliplex kit (Millipore, USA). This kit also tested for osteocalcin and osteoprotegerin. Specifically, 10 μL of lysate were used for this test.

10 readings for each groups were analyzed after testing semi-quantitatively for the presence of Col-I. Immunohistochemistry was used in this test. The disks, previously fixed in 4% formaldehyde, were quenched in 0.6% H₂O₂, blocked in 10% FBS, and a polyclonal anti-Col-I primary antibody (code ab34710, Abcam) was added overnight at a dilution of 1/100. This step was followed by PBS-T washes and the addition of a FITC linked secondary antibody (Code ab96895, Abcam) also using a dilution of 1/100. As in RUNX2, primary to secondary antibody ratio was optimized after performing an unpublished signal-to-noise ratio. The disks were washed again in PBS-T and a drop of ProLong® Gold Antifade with DAPI was added to the bottom of the plate where the disks were inverted for microscopy. A total of 10 intensity readings were obtained from different sections of the disks. Readings were averaged and Col-I was quantified.

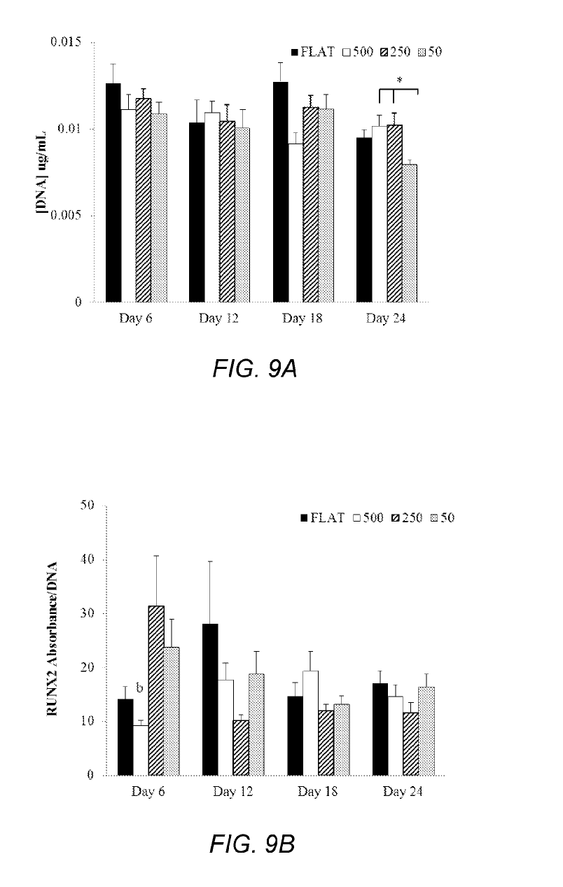

After staining the disks for Col-I and nuclei, the disks were analyzed under fluorescent microscope (SFL7000, Leica) for both Col-I and DAPI. 20× random images were taken of each disk inside the artificial microchannels for both collagen and nuclei. Because the disks had cells growing on a three-dimensional substrate, the pictures of the cells only focused on the valleys of the microchannels. The DAPI pictures were used to determine the angle of orientation of the cells with respect to the microchannel direction. This was done assuming that when the nucleus of the cell was elliptical in shape, the long axis of the nucleus matched the long axis of the cytoskeleton of the cell (Yim, E. K. F., et al., Nanopattern-induced changes in morphology and motility of smooth muscle cells. Biomaterials, 2005. 26(26): p. 5405-5413.) Images were analyzed using Bioquant Osteo system (Bioquant Osteo 2010, Nashville, Tenn.). The angle of the cell was measured in degrees, with 0° being parallel to the microchannel direction, and 1 to 90° and −1 to −90° being of an angle to the right or to the left of the microchannel respectively. A schematic to describe this is shown in FIG. 8. When analyzing the flat control disks, bias measurement orientation due to the media meniscus effect was prevented this was accomplished by taking consequent pictures from the left side to the right side of the disk, as well as from the top to the bottom; 50 cells were measured per image. Successively, the blue channel images from the nuclei and the green channel from the Col-I were merged using Adobe Photoshop®.

The remainder disk from each group was then tested to determine the mechanical properties of the cell's ECM secretions. This was accomplished using a nano-indenter (MTS nano-indenter XP, MTS System Corporation, MN). The fixed disks were epoxyed onto the surface of the nano-indenter holder and kept wet at all times with PBS. The nano indenter was set to indent in the valleys of the microchannels at a rate of 200 μN/min until a 500 μN load was applied. Then, the sample was unloaded. The resulting load-displacement curve was analyzed for stress-strain, toughness, stiffness, and modulus. 200 random readings were taken from each disk.

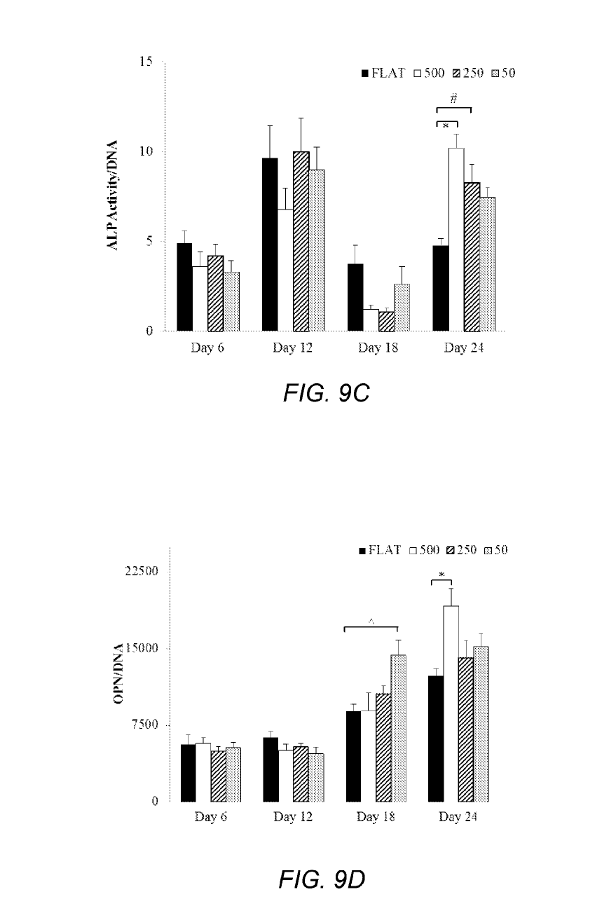

The DNA assay showed that throughout the 24 day experiment the cells did not proliferate consistent with a mature osteoblast phenotype. This is likely to have occurred because all the cells that were seeded in the disks were signaled to differentiate by the dexamethasone found in the osteogenic media. Histogram of the result is shown in FIG. 9A.

The four differentiation markers analyzed were chosen for being indicative of early through late stage osteoblast differentiation. Of the four markers RUNX2 is activated at the earliest time (Zhao, Z., et al., Gene Transfer of the Runx2 Transcription Factor Enhances Osteogenic Activity of Bone Marrow Stromal Cells in Vitro and in Vivo. Mol Ther, 2005. 12(2): p. 247-253). RUNX2 results showed the highest levels at day 6 and then decreased throughout the experiment. The earlier time point shows that the 250 mc and the 50 mc have the highest trend of RUNX2 expression. By day 12 the 250 mc decreased to its lowest level while the control group spiked, showing delayed differentiation. The second chronological differentiation marker is ALP. The data supports this by showing an ALP spike on day 12, and because of its known cyclic activation, a second spike was seen again on day 24 (Oste, L., et al., Time-evolution and reversibility of strontium-induced osteomalacia in chronic renal failure rats. Kidney Int, 2005. 67(3): p. 920-930). At this time point, the 500 mc and the 250 mc were significantly different from the flat control group (P<0.001 and P<0.05 respectively). OPN is chronologically the third differentiation maker of osteoblast stem cells. This data also correlates with our findings and after being minimal on days 6 and 12, they rise on day 18 to have the highest spike at day 24. In particular, the 500 mc had a significantly higher amount of OPN than the rest of the groups on the same 24 day time point (P<0.05). The last differentiation marker tested was DMP1. This marker tests for osteoblast differentiation into osteocytes. The data for this marker shows very slight to no change in time in the expression of this marker. Although possible spikes of DMP1 are seen in the control flat disks at days 12 and 18, and for 500 mc at day 24, none of these changes are significantly different. FIGS. 9B-9E show the histograms with the differentiation markers findings.

A total of 10 readings were analyzed in the quantification of Col-I fluorescence in each group. These findings are shown in FIG. 9F. Although the data was not significantly different, a trend is seen in which all of the tested groups from day 18 produced greater Col-I than the flat control group. The highest values of Col-I were seen at day 18 and slowly decreased by day 24 to roughly the same levels as day 12.

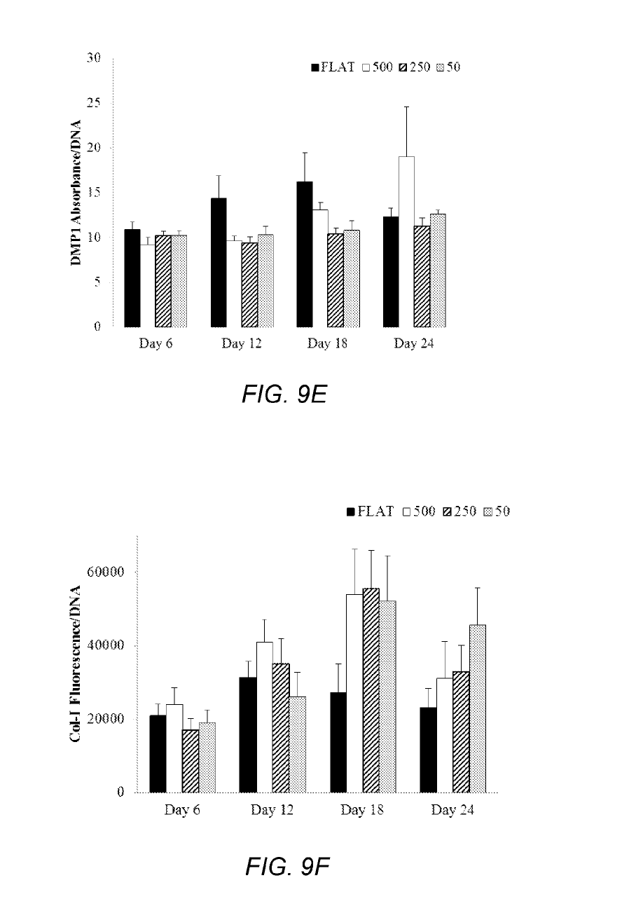

The cell orientation within the microchannels was determined from computational analysis of DAPI stained nuclei. After setting the direction of the microchannels as 0°, fluorescent pictures of the cells were analyzed and the angle of attachment was found. The results show that the cells growing in the flat control group were not able to become organized in 24 days. FIG. 10 shows the resulting frequency distribution of the orientation measured. The narrower the frequency curves, the more organization the cells displayed and vice versa. Also, a frequency curve with a narrow and tall peak at 0° meant that the cells were aligned with the microchannels. Any change in angle meant that cells were organized at a specific angle and so on. Throughout the four time points the frequency distribution shows a flat line. In the earliest time point (6 days) the only group that was more organized than all other groups was 250 mc. The early orientation parallels the microchannels direction. By day 12 the 250 mc still shows organization, although this time there is a slight change in cell orientation to 5°. At the same time the 50 mc starts showing early organization at 0°. The 500 mc is still disorganized after 12 days. At day 18 the 250 mc and the 50 mc remain the most organized ones. The 250 mc orientation shifted a little closer to the 10° angle. The 50 mc remains organized but shows no shift in cell orientation. It is by day 18 that the 500 mc starts to show some level of cellular organization, with most cells pointing towards the 5-15° angle. At day 24 the 50 mc shows a sudden change in organization to 25°. Also the 500 mc shows a higher degree of organization towards the 20° angle. The 250 mc remains stable and does not change orientation, staying around the 10° angle.

In one embodiment, a 3D tissue engineered HAp/TCP platform is formed with longitudinal porous microchannels that simulate the physiological and mechanical properties of cortical bone. In one embodiment, a 3D scaffold of dense longitudinal and interconnected microchannels on a HAp/TCP platform is formed that degrades within a year. The structure includes longitudinal microchannels in HAp scaffolds. An Hap/TCP platform is formed by creating a mold into which to cast the HAp. The molds may be reproducible and to yield identical scaffolds with the same microchannel density and size. The microchannels may be created with polyethylene wires having the desired diameter (based on previous tests). Once the slurry hardens, the polymeric wires burn off in the sintering process leaving the cast structure intact. Microchannels may be analyzed with SEM and a stereoscope for diameter integrity and uniformity. Moreover, the surface area of the 3D HAp platform may be calculated mathematically to determine how many cells can attach to the sides of the microchannel. The surface area may be found by multiplying the circumference by the length of the channels, finding the surface area of each microchannel, and multiplying it by the number of channels in the scaffold for the total surface area.

The TCP/HAp ratio in the slurry is varied to create different degradation profiles. Preferably the HAp/TCP ratio used is such that TCP release from the structure will be completed within a year, matching the natural bone resorption rate. Five different formulations of HAp/TCP have been prepared and tested: ranging from 100:0 to 0:100 HAp:TCP. To test the TCP release from each scaffold, samples are compared to a solid Hap disk scaffold while submerged in simulated body fluid (SBF). Two different formulas of SBF may be utilized. The first SBF has a neutral pH (7.4), similar to physiological conditions in a healthy environment. The second SBF has a slightly acidic pH (5.6) to reflect physiological healing conditions. Once the scaffolds are submerged in SBF, the solute may be analyzed for calcium release during 60 days. Knowing initial calcium concentration in the SBF, and knowing that TCP is resorbed at a much higher rate than HAp, we can assume that all the extra calcium detected will be coming from the TCP. The amount of calcium in solution may be determined using absorbance. The ratio profile of the varying HAp/TCP ratios in which TCP shows resorption within a year may be chosen for the rest of the experiments.

A second variable in this experiment is the porosity of the scaffold. Porosity may be created by adding different concentrations of sucrose to the slurry just before casting. The sucrose will burn off during sintering at 1,230° C. leaving voids, i.e. pores. Porosity is needed to create microchannel interconnectivity, but too much permeability leads to a loss in mechanical integrity. Thus it is necessary to determine which porosity has the best strength to interconnectivity relation. Five different porosities may be tested: 30, 42, 55, 67, and 80% porosity. Porosity may be assessed by experimentally measuring height and diameter of the scaffolds and calculating the total sample volume (Vsample). A pycnometer (AccuPyc 1340 Gas Pycnometer by Micromeritics—Norcross, Ga.) may be used to find the solid volume (Vsolid). Porosity will be calculated using equation 8.

Porosity=(Vₛₐₘₚₗₑ−V_{solid})×100 (Equation 8)

Once the scaffolds' porosity is determined, a group of scaffolds (n=4) are tested for microchannel interconnectivity using the μCT. With the μCT results, the InterConnectivity Index (ICI) can be calculated. Interconnectivity is calculated as the number of connections between pores. The same analysis may be done by embedding the scaffolds in epoxy glue, sectioning it, and performing morphometric analysis. This is the same procedure that was performed to analyze the 2D disk wave profile, discussed earlier. Another group of samples (n=4) may be tested for mechanical compression strength using the MTS Insight Electromechanical Tester (MTS System Corporation, MN). An end capping technique is used and the scaffolds are crushed at a rate of 1 mm/min.

From the collected data, an analysis of strength, TCP resorption and HAp/TCP ratio, as well as the relationship between strength, pore interconnectivity and porosity may be determined. The results will be plotted in a graph similar to FIG. 6. The point where the two lines meet may be used to determine optimal HAp/TCP ratio and scaffold porosity.

In-vitro cell behavior of bone marrow derived stem cells may be studied for their ability to manufacture organized ECM and strengthen the newly created 3D HAp/TCP platform. HFOBs attached on the walls of the microchannels create multiple ECM secreting layers. This effect will form variable collagen organization as a function of curvature, which will naturally reinforce the implant. Furthermore, media flow through the channels will prevent occlusion and will promote uniform cell infiltration within the scaffold.

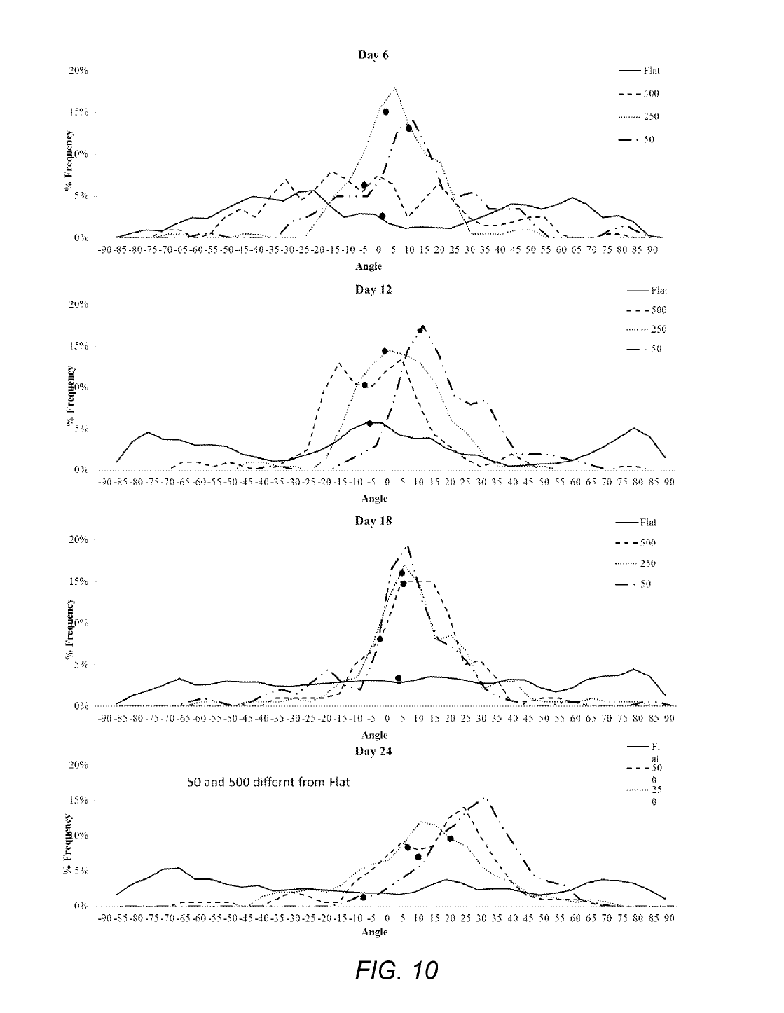

HFOBs may be loaded onto an HAp/TCP substrate and studied for a total of 10 weeks, with a total of five time points at 2, 4, 6, 8, and 10 weeks. High density of HFOBs are seeded on the scaffolds. The HFOBs are allowed to attach to the surface, and at week 1 and week 2 the same amount of cells are seeded. This technique generally yields the highest scaffold cell seeding compared to other similar techniques. The entire study may be performed inside a bioreactor, (FIG. 7), and continuous flow will be created by an IPC High Precision Multichannel Dispenser (Model #CP78001-42—Ismatec, Switzerland). Because bone's internal structure is very complicated, researchers throughout the years have been unable to analyze the fluid flow through an osteon and have relied on theoretical models instead. It is necessary for the cells to be exposed to fluid flow in order to prevent them from occluding the microchannel opening. We hypothesize that without the flow the channels will be sealed off by the cells almost immediately. Also, without flow there would be no infiltration inside the scaffold, leaving a high amount of cells attaching over confluence in the area surrounding the top of the scaffold. The fluid velocity results may be studied with finite element analysis techniques to determine the flow rate. The experimental values for blood flow rate have not been well reported in human bone, however some data is available from canine bone with values ranging from 5 to 11 mL/min/100 g tissue in Tibia and Femur bone respectively. To test the scaffold, cells may be seeded at high density and allowed to attach for 18 hours. After attachment, a pump is turned on. At week 1 and 2 the same procedure may be repeated to bring the scaffold to full confluence. The pump is shut down 18 hours after each seeding. FIG. 11 is a representation of a bioreactor that may be used to test flow rates.

Scaffold cell lysates may also be tested for RUNX-2 activity, ALP activity, and, in some embodiments OC, OP, and ON. Some scaffold may be processed for histology and cut into longitudinal sections and then stained with Sirius Red Collagen. These test may be used to determine collagen fibril orientation. Bioquant® may be used to determine the amount of collagen secreted in each microchannel. After sectioning a scaffold many microchannels will be available to use for readings. In some embodiments, 6 randomly chosen microchannels may be analyzed.

Scaffolds may also be tested for cell morphology. Specifically the orientation of cell attachment and the layering of the cells within the microchannel may be tested. Generally, only 2 scaffolds in this group, and 6 microchannels will be analyzed in each scaffold. All the samples from this group may also be processed for histology. For example, the first sample may be cut into cross sections and the second sample into longitudinal sections. The slides may be stained red using Alizarin Red, which stains for bone (calcium), and counterstained using Aniline Blue which stains Collagen Type I blue. The radial sections will demonstrate how the cells stacked within the microchannel, if they assumed the shape of an osteon, how thick of a cell layer was created and how much of the original, channel was left opened. The longitudinal section may be used to analyze the orientation of the cells to the microchannels using the fractal analysis and cell infiltration within the scaffold as measured by histomorphometry. The relationship of percent infiltration to percent porosity will also be determined.

The mechanical strength of the newly developed scaffolds may also be tested. It is believed that by creating this osteon-resembling platform the cells will use the existing structure to recreate an osteon thus reinforcing the strength of the scaffold. In an embodiment, samples may undergo compression testing and 3 other samples may be assessed for local increases in strength. The compression tests keep the same parameters as specified previously (end-capping, strain rate). Local strength increases may be tested by running the nano-indenter in scratch mode through a cross section of the microchannels. When this is completed, a correlation of porosity to overall strength and ICI, as well as local strength with ICI may be determined:

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A method of forming a scaffold for the repair of load-bearing bone damage comprising:
obtaining a mold having an interior space that is complementary to the bone repair site;
coupling wires to the mold, such that the wires extend through an interior space of the mold
placing a fluid composition of hydroxyapatite and β-tricalcium phosphate into the interior space of the mold; wherein the fluid composition surrounds at least a portion of the wires;
removing the wires from the mold after the fluid composition hardens.

2. The method of claim 1, wherein the wires are formed from a polymeric material and wherein the wires are removed by heating the scaffold to a temperature sufficient to burn off the wires.

3. The method of claim 1, further comprising securing the wires to the mold, such that the position of the wires does not substantially change when the fluid composition is added to the interior space.

4. The method of any one of claim 1, further comprising placing a porous material in the interior space prior to coupling the wires to the mold, wherein the wires pass through the porous material when the wires are coupled to the mold.

5. The method of claim 1, wherein the fluid composition further comprises particles of an organic material, wherein the method further comprises heating the scaffold to a temperature sufficient to decompose the particles of organic material.

6. The method of claim 1, further comprising subjecting the mold to ultrasound, after the fluid composition has been placed in the interior space of the mold.

7. A bone scaffold made by the method of claim 1.

8. A bone scaffold for the repair of load-bearing bone damage comprising hydroxyapatite and β-tricalcium phosphate, wherein the scaffold has an architecture that matches the structure of cortical bone.

9. The scaffold of claim 8, wherein the scaffold has longitudinal microchannels that recreate the structure of secondary osteons.

10. The scaffold of claim 8, wherein the scaffold has high interconnectivity to recreate the structure of Volkman's canals that allows blood and nutrients to be moved within the scaffold.

11. The scaffold of claim 8, wherein the amount of β-tricalcium phosphate present in the scaffold is preselected to cause part of the scaffold to be resorbed in at least a year.

12. The scaffold of claim 8, further comprising stem cells coupled to the scaffold.

13. The scaffold of claim 8, wherein the scaffold has porosity that increases surface area for stem cell adhesion.

14. A mold for forming a bone scaffold for the repair of load-bearing bone damage, comprising:
a body having an interior surface, wherein the interior surface defines the shape of the scaffold;
a top plate and a bottom plate, couplable to a top end and a bottom end of the body, wherein the top plate and the bottom plate each comprise a plurality of openings, wherein, during formation of the scaffold, one of more wires are placed in the openings.

15. The mold of claim 14, further comprising a middle rod, couplable to the top plate and/or the bottom plate, wherein the middle rod defines a hollow portion of the scaffold being formed by use of the mold.

16. The mold of claim 14, further comprising one or more alignment rods, extending from the bottom plate, through the body, to a top plate of the mold.

17. The mold of claim 16, wherein the top plate and/or the bottom plate are movably positioned with respect to the body, by sliding the plates along the alignment rods.
